(19) United States
(12) Patent Application Publication
(10) Pub. No.: US 2010/0317682 A1
(43) Pub. Date: Dec. 16, 2010

(54) Single Dosage Unit Including Opioid and Methylnaltrexone or Methylnaltrexone Salt

(76) Inventors: Richard Fuisz, McLean, VA (US); Joseph M. Fuisz, Washington, DC (US)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/482,828

(22) Filed: Jun. 11, 2009

(51) Int. Cl. A61K 31/439; A61P 25/36

(52) U.S. Cl. 514/282

(57) ABSTRACT

The use of methylnaltrexone bromide in combination and in one dosage unit, with any opioid drug in order to prevent the constipation which can be associated with opioids.

### BACKGROUND OF THE INVENTION

Opioid drugs can cause constipation. This is especially true in the debilitated patient. It is known and published in numerous medical papers that methylnaltrexone bromide acts to block the mu-receptors, which receptors are the causative receptors on which opioids act in the human GI system to cause constipation. It has been known to give methylnaltrexone bromide as a separate medication to those patients who through age and debilitation, are subject to severe constipation when receiving opioid medications.

For example, U.S. Patent Application Publication No. 2004/0259899 A1 to Sanghvi et al. discloses that one side-effect of exogenous opioid use is constipation, and that peripheral opioid antagonists are being tested for relieving such side-effects; it proposes methods for treating constipation that include administration of peripheral opioid antagonists, such as methylnaltrexone, in combination with laxatives and/or stool softeners.

### SUMMARY OF THE INVENTION

The present invention relates to a single dosage unit comprising an opioid and methylnaltrexone or a methylnaltrexone salt, such as methylnaltrexone bromide, for use by a human.

### DETAILED DESCRIPTION OF THE INVENTION

The present invention unites an opioid and methylnaltrexone or preferably a methylnaltrexone salt, preferably methylnaltrexone bromide, into a single dosage unit to obtain the analgesia of opioids without the constipation. The present invention combines methylnaltrexone bromide with any opioid drug into a single dosage unit so as to prevent constipation and yet not interfere with the analgesic effect of the opioid. Since the methylnaltrexone bromide does not cross the blood brain barrier, there is no interference with analgesia.

One aspect of the present invention is a single dosage unit containing an opioid and methylnaltrexone bromide.

The opioid may be one or more opioids including, but not limited to natural opiates, alkaloids contained in the resin of the opium poppy including morphine, codeine and thebaine, but not papaverine and noscapine which have a different mechanism of action; semi-synthetic opiates, created from the natural opioids, such as hydromorphone, hydrocodone, oxycodone, oxymorphone, desomorphine, diacetylmorphine (heroin), nicomorphine, dipropanoylmorphine, benzylmorphine and ethylmorphine; and fully synthetic opioids, such as fentanyl, pethidine, methadone, tramadol and propoxyphene.

The chemical name for methylnaltrexone bromide is (R)—N-(cyclopropylmethyl)noroxymorphone methobromide. The molecular formula is C₂₁H₂₆NO₄Br, and the molecular weight is 436.36. Methylnaltrexone bromide is available under the tradename RELISTOR from Wyeth of Madison, N.J. Methyl naltrexone ((5α)-17-(cyclopropylmethyl)-3,14-dihydroxy-17-methyl-4,5-epoxymorphinanium-17-ium-6-one-chemical formula C₂₁H₂₆NO₄) or other pharmacologically active salts thereof may be used in place of some or all of the methylnaltrexone bromide, but methylnaltrexone bromide is preferred.

The dosage unit can be in the form of an oral dosage unit, a solution for subcutaneous injection or in any other dosage form acceptable to deliver opioid and methylnaltrexone bromide to a patient, especially a human patient.

As a minimal dose for subcutaneous injection, the recommended dose of methylnaltrexone bromide in the single dosage unit is 8 mg for patients weighing 38 to less than 62 kg (84 to less than 136 lb) or 12 mg for patients weighing 62 to 114 kg (136 to 251 lb). Patients whose weights fall outside of these ranges should be dosed at 0.15 mg/kg. While these dosages are for subcutaneous injection, those skilled in the art would be able to calculate the recommended dosage unit for other dosage units in terms of corresponding blood levels.

It will be apparent to those skilled in the art that various modifications and variations can be made in the methods and compositions of the present invention without departing from the spirit or scope of the invention. Thus, it is intended that the present invention cover any and all modifications and variations of this invention that come within the scope of the claims and their equivalents.

We claim:

1. A single dosage unit comprising an opioid and methylnaltrexone bromide for use by a human.

2. A single dosage unit comprising an opioid and a methylnaltrexone salt for use by a human.

3. A single dosage unit comprising an opioid and methylnaltrexone for use by a human.
